(19) United States
(12) Patent Application Publication
(10) Pub. No.: US 2004/0186496 A1
(43) Pub. Date: Sep. 23, 2004

(54) Disposable scalpel

(75) Inventors: Dan Sandel, Chatsworth, CA (US); Ruane Jeter, Chatsworth, CA (US); Noel Gharibian, Chatsworth, CA (US)

Correspondence Address: PERKINS COIE LLP, POST OFFICE BOX 1208, SEATTLE, WA, 98111-1208, US

(73) Assignee: Sandel Medical Industries LLC

(21) Appl. No.: 10/394,131

(22) Filed: Mar. 21, 2003

(51) Int. Cl. A61B 17/32

(52) U.S. Cl. 606/167

(57) ABSTRACT

A disposable scalpel has a blade attached to a handle. The scalpel is weighted, so that although it is disposable, it has a weight similar to the weight of a conventional, nondisposable metal scalpel. The scalpel may be weighted by one or more solid metal slugs within the handle, by particles of weight material within the handle, or by manufacturing the handle including weight materials, such as molded plastics including weighted filler, such as a metal powder. A shield assembly is slidable within the handle from an extended position, where the shield blocks access to the blade, to a retracted position, where the shield is substantially within the handle, and the blade is fully exposed. A locking mechanism locks the shield into the extended and retracted positions.

### BACKGROUND OF THE INVENTION

The field of the invention is disposable scalpels.

Scalpels used in surgical procedures typically have a disposable cutting blade attached to a metal handle. The blade is often attached to the handle via a slot formed in the blade and a corresponding track or lug protruding from the handle. When the blade needs to be changed, the user slides the slot along the track until the blade can be removed and then replaced. However, changing the blade is generally difficult and dangerous. Moreover, the sharp edge of the used blade is a hazard to nurses and doctors during disassembly or disposal.

To avoid having to load, replace, or remove the blade from the handle of a scalpel, one-time-use or disposable scalpels have been used. These disposable scalpels are inexpensively constructed. Typically, the handle of a disposable scalpel is made entirely out of plastic, to reduce manufacturing costs. As a consequence of this construction, disposable scalpels are approximately 80% lighter than reusable scalpels.

However, many surgeons prefer the weight and feel of conventional reusable scalpels. Conventional scalpels, having metal handles, are relatively heavy and feel “solid” in the surgeon's hand. Disposable, lightweight scalpels do not have the often preferred weight and feel of a conventional scalpel. Consequently, many surgeons continue to use conventional medical scalpels despite their drawbacks.

Accordingly, there is a need for a disposable scalpel that has a weight and feel similar to a conventional non-disposable scalpel, and which provides the traditional “solid” feel of a conventional scalpel.

### SUMMARY OF THE INVENTION

To this end, the invention provides a disposable scalpel having a handle and a blade supported by or on the handle. Unlike known disposable scalpels, the scalpel is weighted, so that it has a weight substantially similar to the weight of a conventional, non-disposable scalpel. While the present scalpel is disposable, and inexpensive to manufacture, it still feels “solid,” like a traditional medical scalpel having a metal handle and a separate disposable blade. The weight added to the scalpel may be located at any place on the scalpel, and in various forms.

The handle of the present scalpel can be made (1) by combining a weighted, preferably metallic, e.g., steel, lead, etc., filler with a compatible binder and molding this composition into the desired handle shape via injection molding, compression molding, thermoset molding, or similar techniques; (2) by using one or more weighted, preferably metallic, inserts or solid metal slugs, on a handle, which may otherwise be entirely a plastic or polymeric composition; or (3) by filing a hollow chamber of a handle, which may be entirely plastic or a weighted composition, with a weighted filler such as fine lead particles or a nonmetallic weight equivalent.

The above-described embodiments may be combined with one another to arrive at the desired weight of a conventional, non-disposable scalpel, i.e., these embodiments are not mutually exclusive. As an alternative, the handle may also be solid, rather than hollow, and constructed of a heavy plastic or metal to arrive at the desired end weight. The heavy plastic used to construct the solid handle may be comprised of a weighted polymeric composition.

Another separate aspect of the invention is to provide a weighted, disposable scalpel that includes a sliding shield to block the blade tip or surround the blade when the scalpel is not in use. The shield is within the handle. Consequently, the surgeon's hand grasps only onto the handle, and not the shield. This helps to provide a more solid feel, similar to a reusable scalpel. In this manner, safe handling of the disposable scalpel is promoted. The sliding shield can preferably be permanently locked into an open and/or closed position.

Other and further objects and advantages will appear hereinafter. The invention resides as well in subcombinations of the features described.

### BRIEF DESCRIPTION OF THE DRAWINGS

In the drawings, wherein the same element number indicates the same element in each of the views:

FIGS. 1A and 1B are front, top and left side perspective views of the present disposable scalpel, with the blade shield shown in the extended and withdrawn positions, respectively.

FIG. 2 is a right side view of the scalpel shown in FIG. 1B.

FIGS. 3A and 3B are perspective views of the scalpel shown in FIGS. 1A and 1B, with the cover removed for purposes of illustration.

FIGS. 4A and 4B are perspective views of internal components of the scalpel shown in FIGS. 1A and 1B, with the scalpel body removed for purpose of illustration.

FIG. 5 is a section view taken along line 5-5 of FIG. 1.

FIG. 6 is a front end view of the scalpel shown in FIG. 1.

FIG. 7 is a perspective view of the scalpel body shown in FIG. 1.

FIG. 8 is a perspective view of the cover shown in FIG. 2.

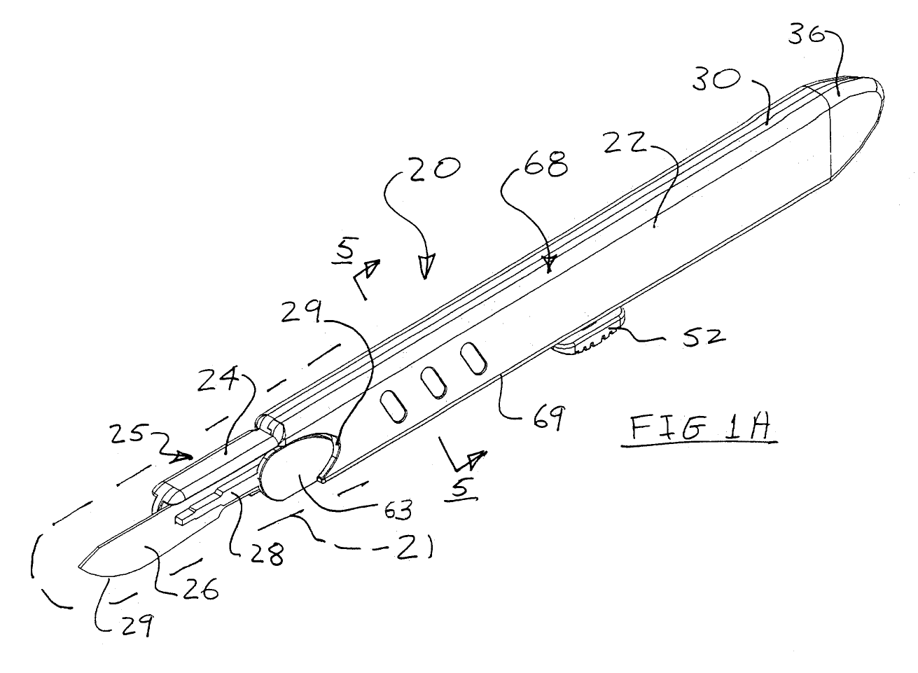

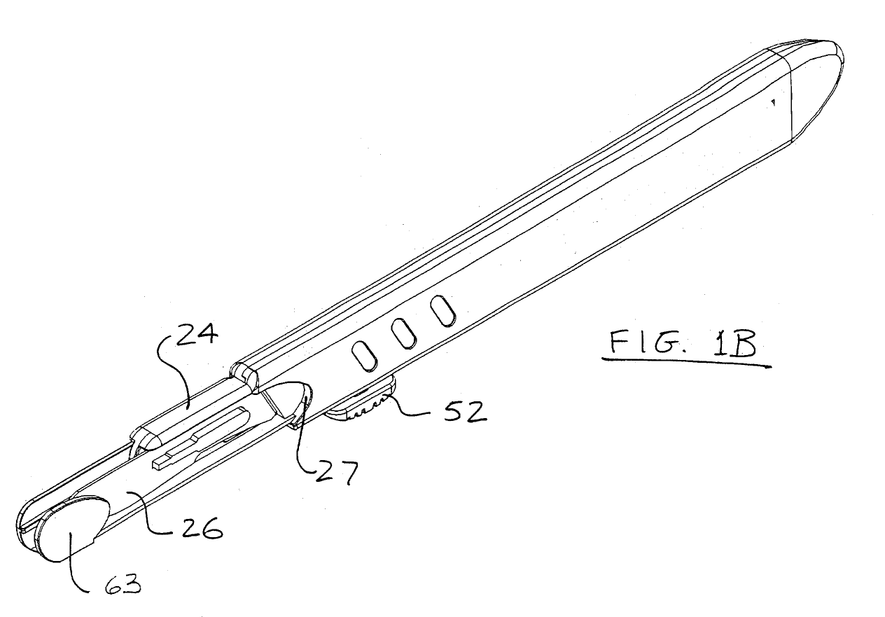

FIGS. 1B, 3B, and 4B are shown at a reduced scale.

### DETAILED OF DESCRIPTION OF THE DRAWINGS

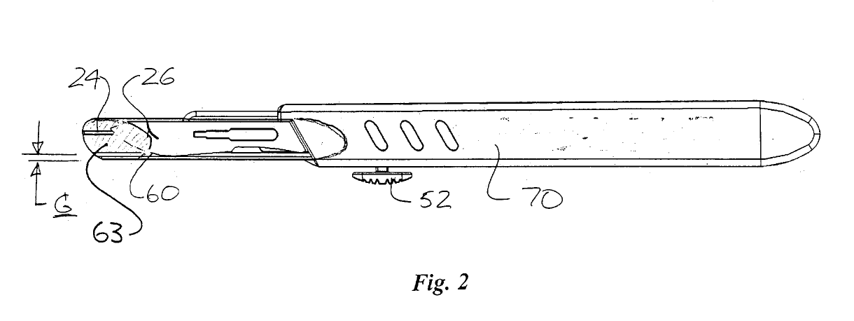

Turning now in detail to the drawings, as shown in FIGS. 1A, 1B and 2, a scalpel 20 has a body 22 including a blade holder 24 at the front end 25. A blade 26 is mounted on a blade lug 28 on the blade holder 24, using well-known techniques. As the scalpel 20 is intended for single use, the blade 26 may be permanently attached to the blade holder 24.

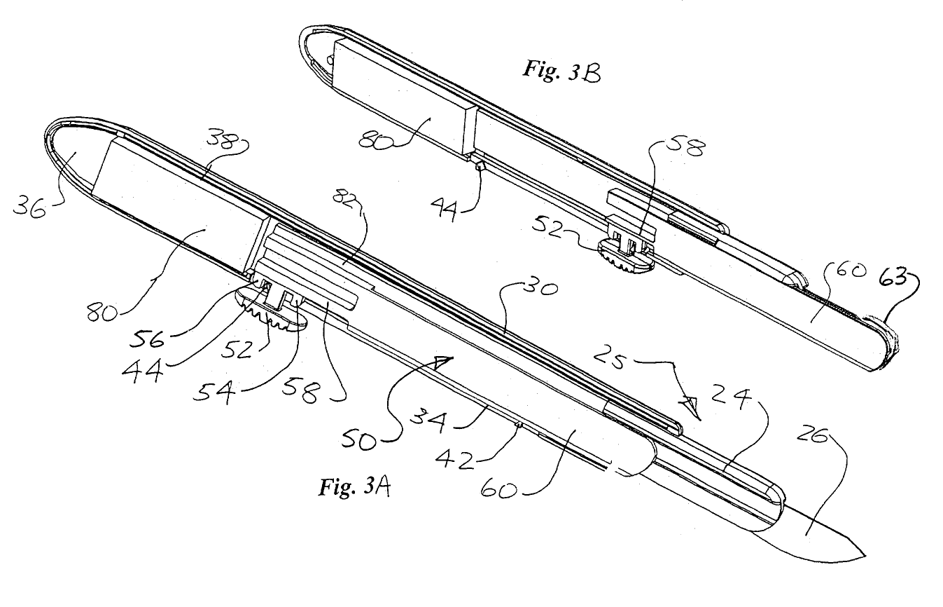

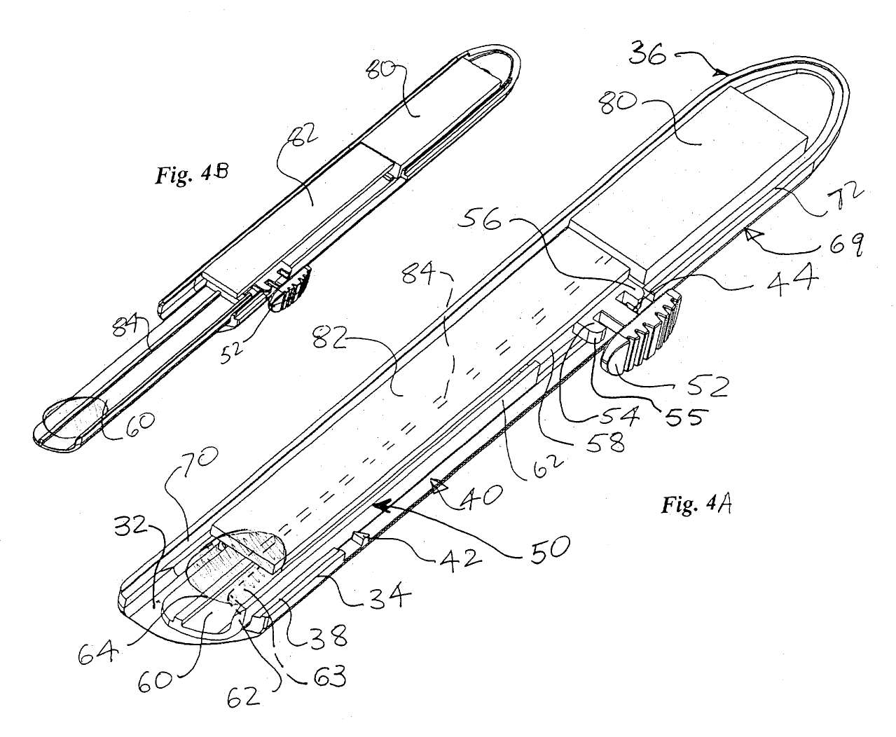

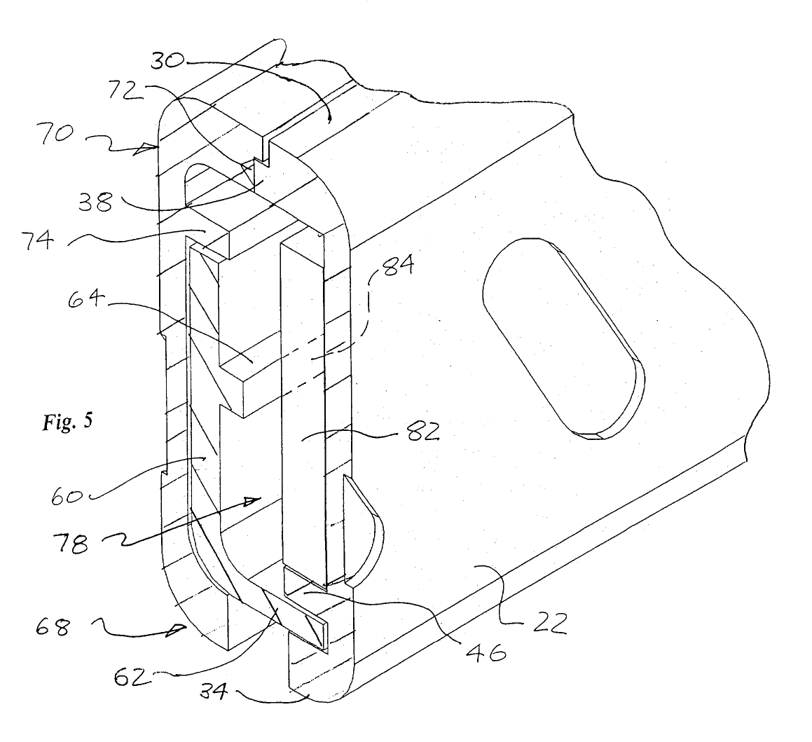

Turning to FIGS. 3A, 3B, 4A and 4B, the body 22 has a top wall 30, a side wall 32, and a bottom wall 34, forming a channel-like cross section, as shown in FIG. 5.

Referring to FIGS. 2-4B, a cover 70 is attached to the body 22, with the cover 70 and body 22 forming a handle for the scalpel 20, containing an interior hollow space generally designated as 78. Referring still to FIG. 5, a rim 38 on the top wall 30 of the body 22 projects into a rim slot 72 in the cover 70, to help align and join the body 22 and cover 70 together.

The body 22 and cover 70 when joined together form the scalpel handle 68. As best shown in FIG. 4, an actuator slot 40 is formed in the bottom surface 69 of the handle 68, preferably via a slot or opening at the bottom surface of each of the body 22 and cover 70. A front latch 42 is located at the front end of the actuator slot 40. Similarly a rear latch 44 is positioned at the back end of the actuator slot 40. The front and rear latches 42 and 44 are preferably formed as triangular lugs or bosses on the body 22 and cover 70.

Referring to FIGS. 3A, 3B and 4, a sliding shield assembly 50 includes an actuator button 52 joined to or integral with a rear finger 58. The activator button 52 has a front hook 54 and a rear hook 56, each having a curved shoulder 55. The rear finger 58 is flexibly or resiliently attached to or part of a side plate 60 of the shield assembly 50. As shown in FIGS. 4A and 4B, a bottom plate 62 is joined preferably perpendicularly to the side plate 60. A second plate 63, shown as an oval or egg-shaped plate, may optionally be provided on the bottom plate 62, extending upwardly, parallel to the side plate 60. Turning momentarily to FIGS. 1A and 1B, the second plate 63 fits into a notch 29 in the body 22, when the shield assembly is in the retracted position shown in FIG. 1A.

Referring still to FIGS. 3A, 3B, 4A and 4B, a rear weight 80, preferably in the form of one or more solid metal slugs or inserts, is provided within the handle 68, behind the actuation slot 40, adjacent to the back end 36 of the scalpel 20. The rear weight 80 is optionally attached to the body 22 during manufacture, to secure it in position, e.g., by molding in place, adhesives or other well-known techniques. Referring momentarily to FIG. 8, a divider wall 76 on the cover 70 may be provided to help locate and secure the rear weight 80 into position.

While a solid metal slug or insert is the preferred form of the rear weight 80, other forms of weight may also be used, including loose particles of heavy materials, such as metals, including lead, iron, and steel. Heavy non-metals may also be used, either as loose particles contained within the handle 68 as a solid slug, or even as a solid molded in section of the handle 68 (i.e., by adding material to the back end of the body 22, the cover 70, or both, or at other places).

Referring still to FIGS. 4A and 4B, a front weight 82 is also preferably included within the hollow space 78 in the handle 68. The front weight 82, while preferably a solid metal slug, may also be provided in the other forms described above in connection with the rear weight 80. In addition, the front weight 82 may be part of, or an extension of, the rear weight 80.

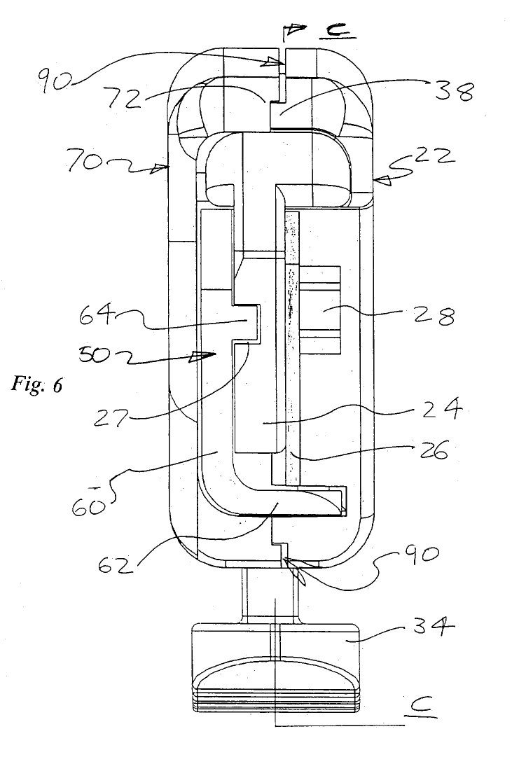

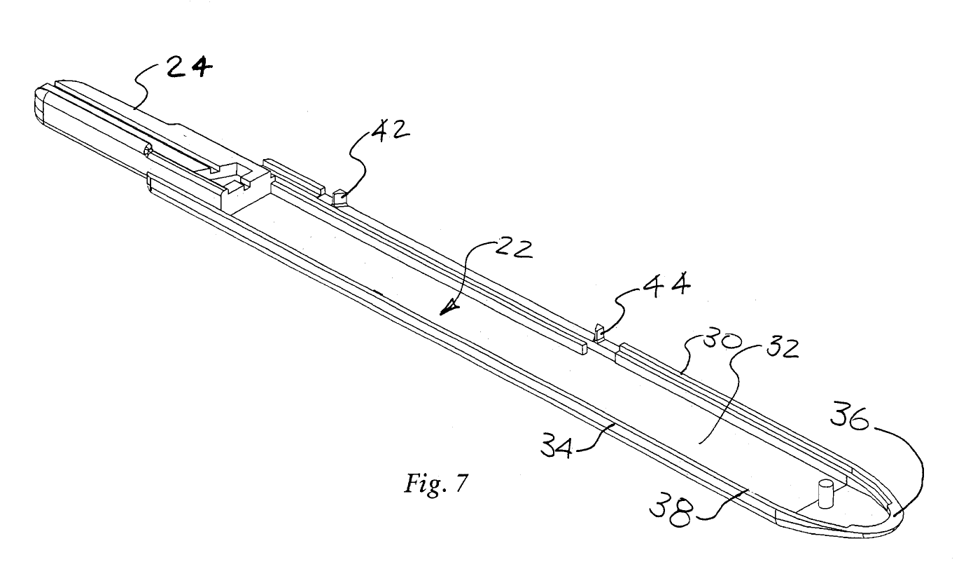

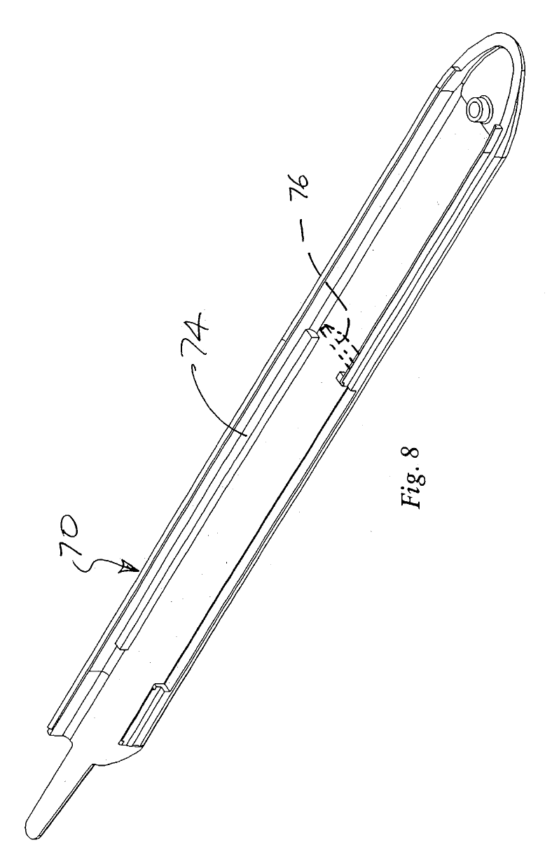

As shown in FIG. 6, a slot 27 extends along one side of the blade holder 24. The tongue 64 in the shield assembly 50 projects into the slot 27. A slot extension 84 may also be provided in the front weight 82 (if used), with the tongue 64 also extending into the slot extension 84. As shown in FIG. 5, a weight shelf 46 may be provided on the body 22, to help position and hold a front weight 82, and also as a secondary guide for the sliding shield assembly 50 (with the bottom plate 62 of the shield assembly 50 extending into a groove formed between the weight shelf 46 and the bottom wall 34 of the body 22). Similarly, as shown in FIGS. 5 and 8, a shield guide 74 is preferably provided on the cover 70. As shown in FIG. 5, with the scalpel 20 assembled, and the body 22 and cover 70 engaged together, the shield guide 74 acts to help position the top edge of the side plate 60.

As shown in FIG. 6, due to the offset lateral or side to side position of the blade holder 24, the blade 26 is positioned near the center line CC of the scalpel 20. As the handle 68 of the scalpel 20 is formed by the body 22 and cover 70, a parting line 90 may be located at the interface between the rim 38 and slot 72, depending on the method of manufacture.

The body 22, cover 70, and shield assembly 50 are preferably made of a molded plastic material. Although the scalpel 20 is intended to be disposable, it has a weight similar to the weight of a conventional reusable metal (typically, stainless steel) scalpel. The weight of the scalpel 20 is preferably 20-35 grams, and more preferably 20-30 grams. Accordingly, although it is disposable, the scalpel 20 provides the feel of a reusable scalpel in the surgeon's hands. Consequently, the surgeon is provided with the feel of a reusable scalpel with the convenience, safety, cost savings, inventory control, and other advantages of a disposable scalpel. Safety aspects are also important to the OR nurse and the housekeeping personnel who handle disposal of sharp instruments.

Any number of techniques can be used to provide the scalpel 20 with the desired weight. Either the front weight 82, the rear weight 80, or both weights (in any form) may be used. For embodiments where the handle 68 comprising the body 22 and cover 70, and optionally the shield assembly 50 as well, are each manufactured as compression or injection molded plastic parts, a separate weight element, such as the weights 80 or 82, may advantageously be used. Alternatively, in an embodiment not requiring any separate weight elements, the handle 68 may be manufactured of a heavy plastic material. These materials include low cost therm-plastic molding resins, such as polyethylene terephthalate (PET) or polybutylene terephthalate (PBT), mixed with a metal powder, such as lead powder. See for example, U.S. Pat. No. 5,665,808, incorporated herein by reference. Thus, the term weight element or weighting means as used here is defined as any element provided or material selected to bring the weight of the scalpel 20 into the desired weight range.

In use, the scalpel 20 is preferably provided in a sterile condition within a package or container 21. The shield assembly 50 will ordinarily be in the extended or front position, as shown in FIG. 2. In this position, the cutting surface 29 of the blade 26 is covered or blocked by the bottom plate 62 of the shield assembly 50, as shown in FIG. 6. As shown in FIGS. 1B and 2, the tip or point of the blade 26 is also shielded by the side plate 60 and the second plate 63. As shown in FIG. 2, with the shield assembly 50 in the extended position, the bottom plate 62 is nominally spaced apart from the cutting edge 29 of the blade 26 by a very small gap G. This prevents the blade 26 from inadvertently coming into contact with any person or object. Referring to FIGS. 1A, 3A and 4A, in use, the shield assembly 50 is in the withdrawn or back position, as shown. The blade 26 is then fully exposed. The handle 68 is continuous on the top and sides, providing the same feel as a reusable scalpel. The actuator button 52 protruding out from the bottom of the handle 68, through the actuator slot 40, is positioned on the bottom to reduce interference with the surgeon's grip on the handle 68.

When the surgeon has completed use of the scalpel 20, or to temporarily shield the blade 26, the actuator button 52 is pushed upwardly slightly, with finger pressure. This causes the rear finger 58 to flex upwardly, or inwardly (towards the top wall 30), allowing the rear hook 56 to move up the angled ramp surface of the latch 44 sufficiently to clear the rear latch 44. The shield assembly 50 is then free to move forward. By pushing forwardly on the actuator button 52, the shield assembly 50 slides from the position shown in FIGS. 1A and 3A to the position shown in FIGS. 1B and 3B. As the actuator button 52 approaches the front latch 42, the rear finger 58 again flexes upwardly allowing the front hook 54 to ride up and over the front latch 42. The shield assembly 50 is then locked in the extended position. To return the shield assembly 50 to the withdrawn position, the actuator button 52 must be pushed up, this time to allow the front tooth 54 to clear the top edge of the top latch 42. The curved shoulder 55 on the front hook 54 and the rear hook 56 allows the hooks 54 and 56 to slide up and over the latches 42 and 44, with reduced sliding force. Movement of the shield assembly 50 is prevented, unless the actuator button 52 is pushed in, by the latch 42 or 44 acting as a stop against the flat facing surface of the hook 54 or 56. Preferably, the rear finger 58 provides sufficient spring force to make an audible sound or click, as the shield assembly 50 is moved into the extended and locked position, or the withdrawn and locked position. Consequently, the surgeon and/or the nurse is provided with an audible indication that the shield assembly 50 is fully moved into its locked position (extended or withdrawn). The audible sound provided by the locking mechanism supplements the tactile feedback provided by the movement of the actuator button 52.

As the shield assembly 50 slides to the extended or withdrawn position, the tongue 64 slides within the slot 27 of the blade holder 24, as shown in FIG. 6. This helps to keep the shield assembly 50 aligned and parallel to the top and bottom surfaces of the handle 68 and the blade holder 24. The weight shelf 46 and shield guide 74 (as well as the optional slot extension 84), all shown in FIG. 5, also help to maintain the shield assembly 50 in place, and in alignment, during and after sliding into position.

As shown in FIGS. 1A, 1B and 5, the only moving component on the handle 64 is the actuator button 52. In addition, the blade 26 is directly attached to the body 22. As a result, the scalpel 20 provides a solid feel in the surgeon's hand, similar to a reusable scalpel. In addition, the weighting techniques described above provide a scalpel weight similar to the weight of a reusable scalpel.

Thus, a novel scalpel has been shown and described. Various modifications and substitutions may be made, without departing from the spirit and scope of the invention. The invention, therefore, should not be limited except by the following claims, and their equivalents.

What is claimed is:

1. A scalpel comprising:
a handle;
a blade having a cutting edge attached to the handle;
a shield slidable within the handle from a first position, where the shield blocks the cutting edge of the blade to a second position, where the blade is exposed; and
weighting means for adding weight to scalpel.

2. The scalpel of claim 1 wherein the weighting means comprises one or more weights within the handle.

3. The scalpel of claim 1 wherein the weighting means comprises a front weight and a rear weight in the handle.

4. The scalpel of claim 1 further comprising an actuator button on the shield, with the actuator button slidable with the shield along a bottom, side or top surface of the handle.

5. The scalpel of claim 1 wherein the shield comprises a rear finger, an actuator button on the rear finger having a front hook and a rear hook, and with the actuator button is resiliently displaceable to engage or release the front or rear hook from a latch on the handle.

6. The scalpel of claim 1 wherein the shield comprises a bottom plate joined to a side plate, and with the bottom plate generally perpendicular to the blade, and a second plate attached to bottom plate and parallel to the side plate.

7. The scalpel of claim 1, the shield comprising locking means for locking the shield in the first position and in the second position.

8. The scalpel of claim 1 with the handle comprising body and a cover attached to the body.

9. A disposable single-use scalpel comprising:
a handle;
a blade attached to the handle; and
weighting means for adding weight to the scalpel.

10. The scalpel of claim 9 wherein the handle comprises plastic.

11. The scalpel of claim 9 wherein the weighting means comprises one or more solid metal weights in the handle.

12. The scalpel of claim 9 wherein the weighting means comprises a weight material provided as an additive to the material of the handle.

13. The scalpel of claim 9 further comprising a hollow space in the handle, and a shield positionable within the hollow space.

14. The scalpel of claim 13 wherein the weighting means comprises one or more solid metal slugs within the hollow space.

15. The scalpel of claim 9 wherein the weighting means is adapted to provide a total scalpel weight of 20-35 grams.

16. The scalpel of claim 9 wherein the handle has a tapered front end.

17. The scalpel of claim 13 further comprising locking means for locking the shield into an open or closed position.

18. The scalpel of claim 17 wherein the locking means provides an audible locking sound upon locking.

19. The scalpel of claim 9 further comprising a blade holder on the handle, and with the blade supported on the blade holder, and with the blade located substantially on a center line of the handle.

20. A single use scalpel comprising:
a handle;
a blade having a cutting edge, attached to the handle;
a shield slidable along the handle; and
at least one metal slug in the handle for adding weight of the scalpel.

21. The scalpel of claim 20 further comprising ramp locking means on the handle for locking the shield against movement.

22. The scalpel of claim 20 further comprising a shield actuation button extending out from a lower surface of the handle, and with the shield actuation button attached to the shield.
